(19) United States
(12) Patent Application Publication
(10) Pub. No.: US 2010/0092432 A1
(43) Pub. Date: Apr. 15, 2010

(54) CELL PREPARATION CONTAINING MULTIPOTENTIAL STEM CELLS ORIGINATING IN FAT TISSUE

(76) Inventors: Takenori Ozaki, Nagoya-shi (JP); Kaoru Yasuda, Nagoya-shi (JP); Shouichi Maruyama, Nagoya-shi (JP); Tokunori Yamamoto, Nagoya-shi (JP); Momokazu Gotoh, Nagoya-shi (JP); Seiichi Matsuo, Nagoya-shi (JP); Yasuo Kitagawa, Nagoya-shi (JP)

Correspondence Address: EDWARDS ANGELL PALMER & DODGE LLP, P.O. BOX 55874, BOSTON, MA, 02205, US

(21) Appl. No.: 12/310,034

(22) PCT Filed: Aug. 7, 2007
(86) PCT No.: PCT/JP2007/065431

§ 371 Date: Oct. 27, 2009

(30) Foreign Application Priority Data
Aug. 8, 2006 (JP) 2006-216234
Feb. 9, 2007 (JP) 2007-030456

(51) Int. Cl. A61K 35/12; A61P 9/10; A61P 13/12; A61P 13/02; A61P 17/02

(52) U.S. Cl. 424/93.7

(57) ABSTRACT

[Problems] To provide a novel use of multipotential stem cells originating in a fat tissue.

[Means for Solving Problems] It is intended to provide a cell preparation which contains multipotential stem cells originating in a fat tissue and is usable for an ischemic disease, impairment of renal function, wound, urinary incontinence or osteoporosis. As the multipotential stem cells originating in a fat tissue, use is made of cells which proliferate in the case of centrifuging cells separated from a fat tissue and culturing the thus sedimented cells (an SVF fraction) under low-serum conditions. In an embodiment, a cell preparation containing the SVF fraction is provided.

### TECHNICAL FIELD

The present invention relates to a cell preparation. More particularly, the present invention relates to a cell preparation effective to treat ischemia diseases, renal dysfunction, wound, urine incontinence or osteoporosis.

### BACKGROUND ART

Attempts are made to reconstruct damaged tissues by using multipotent stem cells capable of differentiating into various cells on a world-wide scale. For example, mesenchymal cells (MSCs) as one of the multipotent stem cells have a potential of differentiating into various cells such as osteocytes, chondrocyte, and cardiomyocyte. Much attention has been paid to clinical applications thereof. Conventionally, multipotent stem cells have generally been collected from the bone marrow. However, the amount of multipotent stem cells contained in the bone marrow is small. When clinical application is considered, in order to obtain a sufficient amount of cells, it may be necessary to collect several hundred milliliters of bone marrows under general anesthesia. Thus, burdens to patients are large. Culture technologies capable of obtaining multipotent stem cells from a small amount of bone marrow have been developed. However, such technologies generally need a large amount of serum (for example, about 10%). This makes it difficult to establish a manufacturing process completely keeping out heterogeneous animal materials, which is important for clinical application. Note here that various possibilities of clinical applications of bone marrow-derived multipotent stem cells have been considered, showing that mesenchymal cells are effective for, for example, a renal ischemia-reperfusion injury (non-patent documents 1 and 2).

Recently, some research groups have reported that adipose tissue is promising as a source of multipotent stem cells (non-patent document 3). Furthermore, it was shown that mesenchymal cells proliferated by culturing cells separated from adipose tissue in 10% FCS-containing culture solution are effective for ameliorating ischemia lesion in the lower limb (non-patent document 4). However, the use of such a large amount as 10% serum would be a problem when clinical application is taken into consideration. On the other hand, Kitagawa et al. have reported that it is possible to prepare a large amount of cell population that shows multipotent from adipose tissue by a simple operation. At the same time, the resultant cells have a potential of differentiating into adipose tissue and are effective for reconstructing adipose tissue (patent document 1).

[Patent document 1] International Publication WO 2006/006692A1

[Non-patent document 1] Am J Physiol Renal Physiol 289: F31-F42, 2005

[Non-patent document 2] Masenchymal Stem Cells Are Renotropic, Helping to Repair the Kidney and Improve Function in Acute Renal Failure. J Am Soc Nephrol: 15 1794-1804, 2004

[Non-patent document 3] Secretion of Angiogenic and Antiapoptotic Factors by Human Adipose Stromal Cells. Circulation 109:1292-1298, 2004

[Non-patent document 4] Circulation. 2004; 109: 656-663

### DISCLOSURE OF THE INVENTION

### Problems to be Solved by the Invention

It is thought that adipose tissue is more promising as a source of multipotent stem cells than the bone marrow because adipose tissues can be collected in a large amount by a simple operation or the collection of adipose tissues gives fewer burdens to patients. The clinical application of adipose tissue has been increasingly expected. Although adipose tissue is a material having a great potential in regenerative medicine in this way, few success cases of reconstructing actual tissues by using adipose tissue-derived multipotent stem cells have been reported to date. Therefore, it has been demanded that the effective application of adipose tissue should be clarified.

It is therefore an object of the present invention to provide a novel application of adipose tissue-derived multipotent stem cells.

### Means for Solving Problems

In order to solve the above-mentioned problems, the present inventors have selected some diseases and examined the efficacy of adipose tissue-derived multipotent stem cells to the selected diseases. As a result, in the graft experiments using lower limb ischemia animal model, renal dysfunction animal model, wound animal model, urine incontinence animal model, and osteoporosis animal model, it has been confirmed that the adipose tissue-derived multipotent stem cells promote the reconstruction of tissues and exhibited high therapeutic effects. From these findings, clinical application of adipose tissue-derived multipotent stem cells in these diseases have been developed. Meanwhile, the present inventors have succeeded in developing a new method of preparing a cell population (SVF fraction) containing adipose tissue-derived multipotent stem cells, and clarified that the SVF fraction has high resistance to freezing and thawing.

The present invention provides the below-mentioned cell preparation and the like mainly based on the above-mentioned results.

[1] A cell preparation containing adipose tissue-derived multipotent stem cells and being usable for ischemia disease, renal dysfunction, wound, urine incontinence or osteoporosis.

[2] The cell preparation described in [1], wherein the adipose tissue-derived multipotent stem cells are cells proliferated when a cell population separated from adipose tissue is cultured under low-serum conditions.

[3] The cell preparation described in [1], wherein the adipose tissue-derived multipotent stem cells are cells constituting a sedimented cell population, which are sedimented when a cell population separated from adipose tissue is centrifuged at 800-1500 rpm for 1-10 minutes, or cells proliferated when the sedimented cell population is cultured under low-serum conditions.

[4] The cell preparation described in [2] or [3], wherein the low-serum conditions are conditions in which a serum concentration in the culture solution is 5% (V/V) or less.

[5] The cell preparation described in [1], including a sedimented cell population (a) or (b), which are cell populations containing the adipose tissue-derived multipotent stem cells:

(a) a sedimented cell population collected as sediments by treating adipose tissue with protease, then subjecting the cell population to filtration, and then centrifuging the filtrate; and

(b) a sedimented cell population collected as sediments by treating adipose tissue with protease, and then centrifuging the adipose tissue without filtration.

[6] The cell preparation described in [5], wherein the protease is collagenase.

[7] The cell preparation described in [5], wherein the centrifugation is carried out under conditions at 800-1500 rpm for 1-10 minutes.

[8] The cell preparation described in any of [1] to [7], wherein the adipose tissue is human adipose tissue.

[9] The cell preparation described in any of [1] to [8], which is in a frozen state.

[10] A method for preparing a sedimented cell population, the method including the following steps (1) to (3):

(1) treating an adipose tissue with protease;

(2) centrifuging the adipose tissue after the above-mentioned step without filtration; and

(3) collecting sediments as a sedimented cell population.

[11] The preparation method described in [10], further including the following step (4):

(4) freezing the collected sedimented cell population.

[12] A use of adipose tissue-derived multipotent stem cells for producing a cell preparation for ischemia disease, renal dysfunction, wound, urine incontinence or osteoporosis.

[13] A use of the sedimented cell population described in claim 5 for producing a cell preparation for ischemia disease, renal dysfunction, wound, urine incontinence or osteoporosis.

[14] A treatment method including: administering adipose tissue-derived multipotent stem cells to a patient with ischemia disease, renal dysfunction, wound, urine incontinence or osteoporosis.

### BEST MODE OF CARRYING OUT THE INVENTION

A first aspect of the present invention relates to a cell preparation applied to certain diseases. The cell preparation of the present invention contains adipose tissue-derived multipotent stem cells. Preferably, the cell preparation of the present invention contains only adipose tissue-derived multipotent stem cells as a cell component. The term “adipose tissue-derived multipotent stem cells” in the present invention denotes multipotent stem cells prepared by using adipose tissue as a starting material. The adipose tissue-derived multipotent stem cells of the present invention is prepared in an isolated state by carrying out one or more steps from separation, purification, culture, concentration, and collection, and the like. The “isolated state” herein denotes a state in which it is taken out from its original environment (i.e., a state constituting a part of the living body), and a state that is different from the original state by artificial, operation.

### (Indicated Diseases)

The cell preparation of the present invention is used for ischemia disease, renal dysfunction, wound, urine incontinence or osteoporosis. In the present invention, the term “for ischemia disease, renal dysfunction, wound, urine incontinence or osteoporosis” denotes that the indicated disease of the cell preparation of the present invention includes ischemia disease, renal dysfunction, wound, urine incontinence and osteoporosis. In other words, the cell preparation of the present invention is used for prophylaxis or treatment of ischemia disease, renal dysfunction, urine incontinence, or osteoporosis, or treatment of wound. Therefore, in general, the cell preparation of the present invention is administered to patients (or potential patients) with ischemia disease, renal dysfunction, urine incontinence or osteoporosis, or patients with wound. However, the cell preparation of the present invention can be also used for the purpose of experiments to confirm and verify the effects thereof.

An ischemia is caused by the stop of a blood flow to organs and tissues or an inadequate flow of blood. When the ischemia term is short, with restart (reperfusion) of a blood flow, the function of the organ is recovered. When the ischemia term is long, with reperfusion, the organ and the like is damaged irreversibly (ischemia reperfusion injury), the organ becomes in a state of dysfunction. A disease caused by such ischemia or ischemia reperfusion is referred to as “ischemia disease.” An example of such diseases includes arteriosclerosis obliterans (e.g., lower limb arteriosclerosis obliterans), an ischemic heart disease (e.g., myocardial infarct, angina pectoris), cerebrovascular disorder (e.g., brain infarction), ischemia disorder in the liver, and the like. One of the indicated diseases of the cell preparation of the present invention is such ischemia diseases. Preferable indicated case is arteriosclerosis obliterans or an ischemic heart disease, and particularly preferable case is arteriosclerosis obliterans.

The “renal dysfunction” in the present invention denotes a state in which renal tissue undergoes some injuries and the kidney fails to carry out original functions. An example of renal dysfunction includes acute renal failure, chronic renal failure, hemolytic uremic syndrome, acute tubular necrosis, interstitial nephritis, acute papillary necrosis, glomerular nephritis, diabetic nephropathy, nephritis accompanying collagen disease, nephritis accompanying angitis, pyelitis, nephrosclerosis, drug-induced renal disorder, disorder accompanying graft, and the like. One of the indicated diseases of the cell preparation of the present invention is such renal dysfunction. Preferable indicated case is acute renal failure or chronic renal failure, and particularly preferable case is acute renal failure.

The “wound” denotes a state in which the body surface tissue has a physical damage. The wound is caused by external factor or internal factor. The wound is classified into cuts, lacerations, puncture wounds, bite wound, contused wound, bruise, abrasions, burn, bedsore, and the like, based on the shapes and factors. The kinds of wounds to which the cell preparation of the present invention is applied are not particularly limited. Furthermore, sites of the wound are not particularly limited.

The “urine incontinence” denotes a state in which the urination function (collection of urine and urination) is not in the normal state and urine leaks regardless of a patient's will. The urine incontinence is classified into true urine incontinence and pseudo-urine incontinence (stress urinary incontinence, urinary urge incontinence, reflex urine incontinence, and the like).

The “osteoporosis” is a disease in which bone mass/bone density are reduced, resulting in bones that are brittle and liable to deform and fracture. Based on the causes, the osteoporosis is classified into primary osteoporosis (involutional osteoporosis and idiopathic osteoporosis) and secondary osteoporosis (osteoporosis caused by certain diseases (rheumatoid arthritis, diabetes, hyperthyroidism, genital insufficiency, and the like) or drugs).

### (Administered Subject, Administration Method)

Subjects to which the cell preparation of the present invention are administered include human or non-human mammalians (pet animals, domestic animal, and experimental animal. Specific examples include mouse, rat, guinea pig, hamster, monkey, cow, pig, goat, sheep, dog, cat, and the like). Preferably, the cell preparation of the present invention is used for human.

The cell preparation of the present invention is preferably administered to an affected site by local infusion. However, the administration route is not limited to this as long as the multipotent stem cell as an effective component in the cell preparation of the present invention is delivered to an affected site. An administration schedule can include once to several times a day, once per two days, or once per three days, and the like. The administration schedule can be formed by considering sex, age, body weight, pathologic conditions, and the like, of a subject (recipient).

### (Preparation Method of Adipose Tissue-Derived Multipotent Stem Cells)

Hereinafter, one example of the methods of preparing adipose tissue-derived multipotent stem cells is described.

(1) Preparation of Population of Cells from Adipose Tissue

Adipose tissue can be obtained from an animal by means such as excision and suck. The term “animal” herein includes human and non-human mammalians (pet animals, domestic animal, and experimental animal. Specifically examples include mouse, rat, guinea pig, hamster, monkey, cow, pig, goat, sheep, dog, cat, and the like).

In order to avoid the problem of immunological rejection, it is preferable that adipose tissue is collected from the same individuals as subjects (recipients) to which the cell preparation of the present invention is to be administered. However, adipose tissue of the same kinds of animals (other animals) or adipose tissue heterogeneous animals may be used.

An example of adipose tissue can include subcutaneous fat, offal fat, intramuscular fat, and inter-muscular fat. Among them, subcutaneous fat is a preferable cell source because it can be collected under local anesthesia in an extremely simple and easy manner and therefore the burden to a patient in collection is small. In general, one kind of adipose tissue is used, but two kinds or more of adipose tissues can be used. Furthermore, adipose tissues (which may not be the same kind of adipose tissue) collected in a plurality of times may be mixed and used in the later operation.

The collection amount of adipose tissue can be determined by considering the kind of donors or kinds of tissue, or the amount of necessary multipotent stem cells. For example, the amount can be from 0.5 g in the case of culture, and the amount of about 200 g in the case where culture is not carried out. When a donor is human, it is preferable that the collection amount at one time is about 1000 g or less by considering a burden to the donor.

The collected adipose tissue is subjected to removal of blood components attached thereto and stripping if necessary and thereafter, subjected to the following enzyme treatment (protease treatment). Note here that by washing adipose tissue with appropriate buffer solution or culture solution, blood components can be removed.

The enzyme treatment is carried out by digesting adipose tissue with protease such as collagenase, trypsin and Dispase. Such an enzyme treatment may be carried out by techniques and conditions that are known to a person skilled in the art (see, for example, R. I. Freshney, Culture of Animal Cells: A Manual of Basic Technique, 4th Edition, A John Wiley & Sones Inc., Publication). Preferably, enzyme treatment is carried out by the below-mentioned techniques and conditions.

A cell population obtained by the above-mentioned enzyme treatment includes multipotent stem cells, endothelial cells, interstitial cells, blood corpuscle cells, and/or precursor cells thereof. The kinds or ratios of the cells constituting the cell population depend upon the origin and kinds of adipose tissue to be used.

### (2) Obtaining of Sedimented Cell Population (SVF Fraction: Stromal Vascular Fractions)

The cell population is then subjected to centrifugation. Sediments obtained by centrifugation are collected as sedimented cell population (also referred to as “SVF fraction” in this specification). The conditions of centrifugation are different depending upon the kinds or amount of cells. The centrifugation is carried out for example, at 800-1500 rpm for 1-10 minutes. Prior to the centrifugation, cell population after enzyme treatment can be subjected to filtration and tissue that has not been digested with enzyme contained therein can be removed. For filtration, for example, a filter with a hole diameter of 100-2000 μm, preferably a filter with a hole diameter of 100 μm is used when culture is carried out and a filter with a hole diameter of 250-2000 μm is used when culture is not carried out.

The “sedimented cell population (SVF fraction)” obtained herein includes multipotent stem cells, endothelial cells, interstitial cells, blood corpuscle cells, and/or precursor cells thereof. The kinds or ratio of cells constituting the sedimented cell population depend upon the origin and kinds of adipose tissue to be used, conditions of the enzyme treatment, and the like. The SVF fraction is characterized by including CD34 positive and CD45 negative cell population, and that CD34 positive and CD45 negative cell population (International Publication WO2006/006692A1).

### (3) Low-Serum Culture (Selective Culture in Low Serum Medium)

In this process, the sedimented cell population is cultured under low-serum conditions, and thereby the intended multipotent stem cells are selectively proliferated. Since the amount of serum to be used is small in the low-serum culture method, it is possible to use the serum of the subjects (recipients) themselves to which the cell preparation of the present invention is administered. That is to say, culture using autoserum can be carried. By using autoserum, it is possible to provide a cell preparation capable of excluding heterogeneous animal materials from manufacturing process and being expected to have high safety and high therapeutic effect.

The “under low-serum conditions” herein denotes conditions in which a medium contains not more than 5% serum. Preferably, the sedimented cell population is cultured in a culture solution containing not more than 2% (V/V) serum. More preferably, the sedimented cell population is cultured in a culture solution containing not more than 2% (V/V) serum and 1-100 ng/ml of fibroblast growth factor −2.

The serum is not limited to fetal bovine serum. Human serum, sheep serum, and the like, can be used. Preferably, the human serum, more preferably the serum of a subject to whom the cell preparation of the present invention is to be administered (that is to say, autoserum) is used.

As the medium, a medium for culturing animal cells can be used on condition that the amount of serum contained in the use is low. For example, Dulbecco's modified Eagle's Medium (DMEM) (NISSUI PHARMACEUTICAL, etc.), α-MEM (Dainippon Seiyaku, etc.), DMED: Ham's:F12 mixed medium (1:1) (Dainippon Seiyaku etc.), Ham's F12 medium (Dainippon Seiyaku, etc.), MCDB201 medium (Research Institute for the Functional Peptides), and the like, can be used.

By culturing by the above-mentioned method, multipotent stem cells can be selectively proliferated. Furthermore, since the multipotent stem cells proliferated in the above-mentioned culture conditions have a high proliferation activity, it is possible to easily prepare cells necessary in number for the cell preparation of the present invention by subculture.

Note here that cells selectively proliferated by low-serum culture of SVF fraction is CD13, CD90 and CD105 positive and CD31, CD34, CD45, CD106 and CD117 negative (International Publication WO2006/006692A1).

### (4) Collection of Cells

The cells selectively proliferated by the above-mentioned low-serum culture are collected. The cells may be collected by routine procedures and, for example, collected easily by enzyme treatment (treatment with trypsin or Dispase) and then cells are scraped out by using a cell scraper, a pipette, or the like. Furthermore, when sheet culture is carried out by using a commercially available temperature sensitive culture dish, cells may be collected in a sheet shape without carrying out enzyme treatment.

### (5) Pharmaceutically Preparation

The collected multipotent stem cells are suspended in physiologic saline or a suitable buffer solution (for example, a phosphate buffer solution) and the like, and thereby cell preparation can be obtained. In order to exhibit desirable therapeutic effect, for example, 1×10⁶ to 1×10⁸ cells per dosage may be contained in cells. The contents of cells can be appropriately adjusted by considering sex, age, and weight of subject to be administered (recipient), condition of an affected site, a state of cells, and the like.

Besides the multipotent stem cells, the preparation may include, for example, dimethylsulfoxide (DMSO), serum albumin, and the like, for protecting the cells; antibiotic and the like for inhibiting contamination of bacteria; vitamins, cytokine, and the like, for activating cells, promoting differentiation. Furthermore, the cell preparation of the present invention may contain pharmaceutically acceptable other components (for example, carrier, excipient, disintegrating agents, buffer, emulsifier, suspension, soothing agent, stabilizer, preservatives, antiseptic, physiologic saline, etc.).

In the above-mentioned method, the cell preparation is formed by using cells proliferated by low-serum culture of SVF fraction. However, cell preparations may be directly formed by the low-serum culture of cell population obtained from adipose tissue (without carrying out centrifugation for obtaining SVF fraction). That is to say, in one embodiment of the present invention, a cell preparation including cells proliferated by the low-serum culture of cell population obtained from adipose tissue as an effective ingredient is provided.

In one embodiment of the present invention, a cell preparation is produced by using not multipotent stem cells obtained by selective culture ((4) and (5) above) but SVF fraction as it is (containing adipose tissue-derived multipotent stem cells). Therefore, the cell preparation in this embodiment contains (a) a sedimented cell population (SVF fraction) of sediments obtained by treating subjecting adipose tissue to protease treatment, then subjecting to filtration, and then subjecting the filtrate to centrifugation; or (b) a sedimented cell population (SVF fraction) of sediments obtained by subjecting adipose tissue to protease treatment, and then to centrifugation without filtration processing.

Note here that “using . . . as it is” herein denotes using as an effective components of cell preparation without selective culture.

When the SVF fraction and cells obtained by selectively culturing the SVF fraction (multipotent stem cells) are compared with each other, the SVF fraction has many advantages: (1) time necessary for preparation is short, (2) cost necessary for preparation is small, (3) risk of canceration or infection is small because culturing is not carried out, (4) since it is non-uniform (heterogeneous) cell population, it is advantageous for reconstructing tissue, (5) since it is less differentiated cell population, it is expected to be differentiated into cells suitable for the tissue to be transplanted after transplantation.

The present inventors have examined resistance to freezing/thawing of the SVF fraction (see, the below-mentioned Example). As a result, cell proliferation potency, cytokine secretion capacity, and cell surface antigen are not substantially affected by freezing/thawing. That is to say, the SVF fraction shows high resistance to the freezing/thawing process. In other words, it is found that the SVF fraction can be frozen and stored without substantial change of the property. Based on the finding, when treatment with cell preparation is repeated (twice or more), it is not necessary to collect adipose tissue every time the treatment is carried out. Burdens to patients and operators are reduced, and time, cost and labor necessary for preparation are also reduced.

In one embodiment of the present invention, based on the above-mentioned findings, as the SVF fraction constituting cell preparation, frozen and stored one is used. Furthermore, another embodiment of the present invention provides cell preparation itself in a frozen state.

The present inventors have investigated the preparation method of the SVF fraction (see, the below-mentioned Example). That is to say, they compared a preparation method in which adipose tissue is treated with protease, then filtrated, and centrifuged (conventional method) with a preparation method in which adipose tissue is treated with protease, and then centrifuged without filtration (improved method). As a result, it is shown that the improved method permits obtaining more cells, and sedimented cell population obtained by both methods exhibit excellent therapeutic effects. Thus, it is shown that the improved method is excellent. According to the improved method, the preparation time can be shortened and problem of contamination accompanying the filtration can be avoided.

As another aspect of the present invention, a novel preparation method of SVF fraction is provided based on the above-mentioned findings of resistance with respect to freezing-thawing and the above-mentioned findings of preparation method of SVF fraction. In the preparation method of the present invention, the collected adipose tissue is treated with protease, and to centrifuged without filtration, thus collecting sediments as a sedimented cell population (SVF fraction). The conditions of the centrifugation includes, for example, for 1-10 minutes at 800-1500 rpm. In one embodiment of the preparation method of the present invention, the collected sedimented cell population (SVF fraction) is frozen and the frozen sedimented cell population is obtained. As the “frozen” conditions herein, conditions for freezing cells at, for example, −180° C. or less and preferably −196° C. or less can be employed.

Another aspect of the present invention, the adipose tissue-derived multipotent stem cells or the SVF fraction is used for drug screening, which affects adipose tissue or blood fat. For example, drug screening can be carried out by using an amount of good materials secreted from the fat as an indication. Specifically, the adipose tissue-derived multipotent stem cells or SVF fractions are cultured under the conditions of test material, and then the production amount of Adiponectin (good material secreted from adipocyte, which reduces when the offal fat increases. Furthermore, the production amount of the material which is involved in repair of damage of the blood vessel, and which is useful for delaying the progress of metabolic syndrome, arteriosclerosis, or cancer) is evaluated. This evaluation system is effective for finding drugs exhibiting an effect of increasing and promoting good adipose.

Furthermore, adipose tissue-derived multipotent stem cells or SVF fractions are cultured in the presence of the test material, and the effect/influence of the test material on the cell proliferation rate is evaluated. This evaluation system is effective for finding drugs exhibiting an effect of increasing or suppressing the increase of adipose.

An example of the test material includes organic compounds having various molecular sizes (nucleic acid, peptide, protein, lipid (simple lipid, complex lipid (phosphoglyceride, sphingolipid, glycosylglyceride, cerebroside, etc), prostaglandin, isoprenoid, terpene, steroid, etc.)) or inorganic compounds. The test materials may be derived from natural product or may be synthesized. In the latter case, for example, a combinatorial synthesis technology is used so as to construct an efficient screening system. A cell extract, culture supernatant, and the like may be used as a test material.

### Example 1

### Preparation of Adipose-Derived Multipotent Stem Cell

1. Preparation of Sedimented Cell Population (SVF Fraction) from Adipose Tissue

An SVF fraction was prepared from human adipose tissue by the following procedure.

(1) From a male human (age: 22), the subcutaneous fat was excised with a surgical knife during surgery and collected.

(2) The adipose tissue was washed with 30 ml of DMEM/F12 solution (a medium (Sigma) mixing an equal amount of Dulbecco's Modified Eagle Medium and F12 medium) three times so as to remove the attached blood and the like.

(3) In a sterilized culture dish, the adipose tissue was cut into pieces with a surgical knife.

(4) The adipose tissue was placed in 50 ml centrifugal tube (Falcon), and the weight thereof was measured (about 1 g).

(5) 2 ml of 1 mg/ml collagenase type 1 (Worthington) solution was placed in the above-mentioned centrifugal tube, and then shaken under the conditions at 37° C. at 120 times/min for one hour.

(6) Subsequently, 10 ml of DMEM/F12 solution was placed in a centrifugal tube and subjected to pipetting.

(7) Cell suspension after pipetting was filtrated through a filter (Falcon) having a hole diameter of 100 μm.

(8) The obtained filtrate was centrifuged at ordinary temperature at 1200 rpm for 5 minutes. The sediments were collected as an SVF fraction.

### 2. Low-Serum Culture of SVF Fraction

The SVF fraction was subjected to low-serum culture by the following procedure.

(1) Nucleated cells (3.8×10⁵) in the SVF fraction were suspended in a low-serum culture solution and planted in a fibronectin-coated flask (25 cm) (Falcon). The low-serum culture solution was prepared as follows (a-e).

(a) DMEM (NISSUI PHARMACEUTICAL) (5.7 g), MCDB201 (Sigma) (7 g), L-glutamine (Sigma) (0.35 g), NaHCO₃ (Sigma-Aldrich Japan) (1.2 g), 0.1 mM ascorbic acid (Wako Pure Chemical) (1 ml), and antibiotic (100,000 units/ml penicillin and 100 mg/ml streptomycin) (0.5 ml) were dissolved in 980 ml of distilled water.

(b) The solution was adjusted to pH 7.2 by using 10N NaOH.

(c) The solution was filtrated and sterilized.

(d) 10 ml of linolic acid-albumin (Sigma) and 10 ml of 100×ITS (insulin (10 mg), transferring (5.5 mg), sodium selenite (5 μg, Sigma) were added.

(e) 100 μg/ml bFGF (PeproTech) (1 μl) was added (final concentration: 10 ng/ml) was added.

(2) Total quantity of medium was substituted every two days.

(3) When it reached confluent, it was washed with PBS containing 1 mM EDTA, then, treated with 0.05-0.25% trypsin solution so that cells were exfoliated and collected. The collected cells were similarly planted on a fibronectin-coated plate (produced by using human fibronectin (Sigma)) at the density of 8×10³ cells/cm².

(4) The above-mentioned sub-culture was repeated as needed (in the following experiment, cells after five or six passages were used).

Adipose tissue-derived multipotent stem cells were prepared also from the subcutaneous fat of F344 rat (obtained from Japan SLC) by the same method (low-serum culture after preparation of SVF fraction).

### Example 2

### Effect of Human Adipose Tissue-Derived Multipotent Stem Cells on Lower Limb Ischemia

### 1. Production of Lower Limb Ischemia Model

In a region from the left leg to a femoral region of a 10-week old female CB-17 SCID mouse (CLEA Japan), hairs were removed by using a hair remover cream. The skin of the hair-removed portion was excised, and the left femoral artery was ligated and separated to obtain a mouse lower limb ischemia model. In this model, the lower limb underwent necrosis and dropped off at high rate.

### 2. Experiment (Treatment) Protocol

(1) Human adipose tissue-derived multipotent stem cells (6.7×10⁶) that had been prepared by the method in Example 1 were suspended in 300 μl of DMEM medium (Sigma) and the suspension was injected into the muscle of the left thigh and the lower thigh of the mouse lower limb ischemia model (treatment group). In the control group, only a DMEM medium was infused under the same conditions.

(2) After treatment, the necrosis and deciduation of the left lower limb was observed over time. A case in which the bone was exposed due to deciduation or necrosis of a part of the left lower limb was judged to be lower limb death.

### 3. Result

The cumulative survival rates of lower limbs in the treatment group and control group are shown in FIG. 1. As shown in a graph of FIG. 1, in the treatment group, the obvious improvement of the survival rate of the lower limb is observed. FIG. 2 shows the state of each model (representative example) on day 7 after treatment. The control group shows black necrosis in the left lower limb but the treatment group shows ruddy complexion.

As mentioned above, an experiment in which the mouse lower limb ischemia model was treated with adipose tissue-derived multipotent stem cells was carried out, in the treatment group, obvious improvement of the survival rate of the lower limb is observed. This result shows that treatment with the adipose tissue-derived multipotent stem cells was effective in treatment of the lower limb ischemia lesion.

### Example 3

### Effect of Human Adipose Tissue-Derived Multipotent Stem Cells on Renal Failure 1

### 1. Production of Rat Acute Renal Failure Model

To a 16-week old male nude rat (available from CLEA Japan), 250 mg/kg of folic acid was intraperitoneally administered to form a rat acute renal failure model. This folic acid renal failure model is an acute renal failure model with acute renal tubule disorder, which is an established model from various reports. In this model, it is reported that chronic disorder such as fibrosis remains in a part of the interstitial tissue after the renal function is improved (FIG. 3).

### 2. Experiment (Treatment) Protocol

(1) Human adipose tissue-derived multipotent stem cells (3.8×10⁶) that had been prepared by the method in Example 1 were suspended in 2.0 ml of physiologic saline and the suspension was administered to the rat acute renal failure model from the left internal carotid artery (treatment group). At this time, it was devised that a catheter was inserted from the internal carotid artery to administer cells into the descending aorta so that the cells can reach the kidney more easily. In the control group, an equal amount of physiologic saline was administered under the same conditions.

(2) On days 0, 1, 2, 4, and 13 after the above-mentioned procedure, the blood was collected and blood urea nitrogen (BUN) was measured.

(3) On day 13 after the above-mentioned procedure, the rat was sacrificed and renal tissue was collected. Then, the renal tissue was evaluated by PAS staining and Masson trichrome staining.

### 3. Result

The measurement results of the blood urea nitrogen are shown in FIG. 4. In the treatment group, significant improvement of the renal function is observed. The results of PAS staining and Masson trichrome staining are shown in FIGS. 5 and 6. In the control group, the expansion of the renal tubule and deciduation of the renal tubule epithelium cells are observed. In the treatment group, such images are hardly observed (PAS staining). Furthermore, in the control group, the atrophy of the renal tubule and fibrosis of the interstitial tissue are observed. However, such findings are hardly observed in the treatment group (Masson trichrome staining).

As mentioned above, an experiment in which a rat acute renal failure model was treated with adipose tissue-derived multipotent stem cells was carried out, in the treatment group, obvious improvement of the renal function is observed. Furthermore, chronic renal disorder (fibrosis of the renal interstitial tissue) remaining after acute renal failure is healed is reduced in the treatment group. From the above-mentioned result, it is shown that the treatment with adipose tissue-derived multipotent stem cells is effective for acute renal failure.

### Example 4

### Effect of Human Adipose Tissue-Derived Multipotent Stem Cells on Renal Failure 2

### 1. Production of Rat Acute Renal Failure Model

From a 14-week old male nude rat (available from CLEA Japan), right kidney was extracted. A week after, 200 mg/kg of folic acid was administered from the caudal vein so as to produce an acute renal failure model.

### 2. Experiment (Treatment) Protocol

(1) Seven hours after the administration of folic acid, human adipose tissue-derived multipotent stem cells (4.0×10⁶) that had been prepared by the method in Example 1 were injected into the left renicapsule of a rat acute renal failure model (treatment group). In the control group, only physiologic saline was administered.

(2) On days 0, 1, 2, 6, and 14 after the above-mentioned procedure, the blood was collected and blood urea nitrogen (BUN) was measured.

(3) On day 3 after the above-mentioned procedure, the blood flow in the capillary blood vessel around the renal tubule was measured by using a pencil type CCD camera (FIGS. 7 to 9).

(4) On day 14 after the above-mentioned procedure, the rat was sacrificed and renal tissue was collected. Then, immunostaining was carried out by using a human-specific antibody.

### 3. Result

The measurement result of the blood urea nitrogen is shown in FIG. 10. In the treatment group, significant improvement of the renal function is observed. Furthermore, the result of immunostaining (FIG. 11) shows that the administered cells are not moved into the parenchyma of kidney and the cells are survived under the renicapsule. The collection of the renal tissue and the immunostaining treatment were also carried out a month after and three months after the treatment. As a result, it is shown that the administered cells survive under the renicapsule over the long time (FIGS. 12 and 13). FIG. 12 shows the result of immunostaining one month after the treatment, and FIG. 13 shows the result of immunostaining three months after the treatment. It is shown that the administered cells survive also after three months after the treatment.

As mentioned above, in the treatment group, the administered cells survive well under the renicapsule to ameliorate the folic acid nephropathy. From this result, it is shown that the treatment with adipose tissue-derived multipotent stem cells is effective for the acute renal failure.

On the other hand, as shown in FIG. 14, in the treatment group, the blood flow of the capillary blood vessels around the renal tubule was significantly fast. It is thought that NO in the kidney is increased by cytokine such as VEGF secreted by the injected cells and the blood vessel was expanded and then, the blood flow was increased.

### Example 5

### Effect of Rat Adipose Tissue-Derived Multipotent Stem Cells on Wound

### 1. Production of Rat Skin Defect Model (FIG. 15)

The back of a 7-week old male F344 rat, hairs were removed by using a hair remover cream. Vinyl chloride having a size of 1.5 cm×1.5 cm and the thickness of 0.45 mm was placed on substantially the central portion of the hair-removed portion and marked. After it was disinfected with povidone iodine, the total layer of skin was excised along the marking to thus form a rat skin defect model was obtained.

### 2. Experiment (Treatment) Protocol

(1) Multipotent stem cells derived from F344 rat subcutaneous fat (1.1×10⁷) that had been prepared by the method in Example 1 were suspended in a DMEM medium (Sigma) so that the total amount was 800 μl, and the suspension was injected to the subcutis around the excised skin of a rat skin defect model by using a 26 G injection needle (low-serum treatment group). Thereafter, tegaderm (product of 3M) was patched to the wounded portion. A group in which cells obtained by culturing nucleated cells in the SVF fraction prepared from the subcutaneous fat of the F344 rat in high-serum conditions (EMEM containing 20% FBS was used) (high-serum cultured cells) was compared with a group to which only a DMEM medium was injected under the same condition to each other.

(2) On days 0, 2, 7, 14 and 18 after treatment, an area of the wounded portion was measured. The method for measuring the area was as follows. Firstly, 0.45 mm-thick vinyl chloride sheet was applied to the wounded portion and the edge of the wounded portion was provided with marking, followed by punching the sheet along the marking. The weight of the punched vinyl chloride sheet was measured, and the measured value was converted into the area.

(3) Furthermore, the skin tissue three days after the treatment was collected, and the concentrations of VEGF and VHGF in the tissue were measured by an ELISA method.

### 3. Result

The change of the skin defect area of each group was compared with each other in a graph of FIG. 16. Furthermore, the state of the wounded portion on day 14 after the treatment is shown in FIG. 17. Later than the first week, significant improvement of the skin defect area was observed in the low-serum treatment group (right upper picture) as compared with the control group (left upper picture). Furthermore, as is apparent from FIG. 17, in the treatment group, rapid healing of the wound advances and the state of the scar tissue is excellent. When the low-serum treatment group (right upper picture) and the high-serum treatment group (left lower picture) are compared with each other, a higher effect for promoting the wound healing is observed in the former group.

On the other hand, as shown in the graph of FIG. 18, in the low-serum treatment group, as compared with the control group, the significant increase in the VEGF concentration in the wounded tissue was observed. The HGF concentration was not different between two groups. From the result of immunostaining of the wounded portion (not shown), the low-serum treatment group shows that the infused cells remain in the subcutis even 14 days after the treatment and the cells were not differentiated into the blood vessel.

As mentioned above, when the experiment of treating rat skin defect models by using adipose tissue-derived multipotent stem cells was carried out, in the low-serum treatment group, it was shown that the wound healing was promoted significantly. From the above-mentioned results, it is shown that the treatment with the adipose tissue-derived multipotent stem cells is effective for healing the wound. Furthermore, it is shown that the adipose tissue-derived multipotent stem cells exhibit the higher effect of promoting healing wound as compared with cells cultured in high-serum conditions.

### Example 6

### Cytokine Secretion Capacity of Human Adipose Tissue-Derived Multipotent Stem Cells

### 1. Materials and Method of Experiment

Human adipose tissue-derived SVF fractions were cultured in three kinds of culture solutions: high-serum culture solution (DMEM containing 20% FBS), bFGF-added high-serum culture solution (DMEM containing 20% FBS and bFGF (10 ng/ml)) and low-serum culture solution (low-serum culture solution containing bFGF (10 ng/ml) used in Example 1). Cytokine in the supernatant was measured by an ELISA method. As a control group, human renal fibroblast (HEK293) was used. In experiments, 4-5 passages of sub-cultured cells were used. Furthermore, culture was carried out in 25 cm² flask using 5 ml of culture solution.

Each culture solution was removed by sucking in a semi-confluent state, washed with PBS twice, and then cultured in DMEM containing 10% FBS for 24 hours. At this time, two groups of normal oxygen and low oxygen (1% O₂) are made. This is carried out for examining whether or not cytokine secretion is kept even in the low-oxygen environment assuming that ischemia tissue is treated with cells. After 24 hours, culture supernatant was collected, and cytokine was measured by an ELISA method. At the same time, cells are exfoliated with trypsin and the number of cells was counted. Comparison was carried out based on the secretion amount of cytokine per 10⁶ cells.

### 2. Result

As shown in FIGS. 19 and 20, the low-serum culture group secretes more growth factors as compared with the control group. Furthermore, in the low-serum culture group, as compared with the high-serum culture group and bFGF added high-serum culture group, the secretion amount of VEGF-A (FIG. 21), FGF-7 (KGF) (FIG. 22) and FGF-2 (FIG. 23) are larger. In the low-oxygen environment, the secretion amount of VEGF-A is radically increased. Other cytokines show substantially the same secretion amount as that in the normal oxygen environment. Meanwhile, the secretion amount of VEGF-C and the secretion amount of HGF are not different among groups (FIG. 24). The low-serum culture group also secretes TGF-β, IL-6, IL-10 and IL-8. The secretion amount is larger than those of high-serum group and the bFGF-added high-serum culture group (FIG. 25).

As mentioned above, cells obtained by low-serum culturing adipose tissue-derived SVF fraction exhibit higher cytokine secretion capacity as compared with the cells obtained by a conventional culture method. That is to say, it is clarified that the low-serum culture makes it possible to selectively separate and proliferate cells whose cytokine secretion capacity is extremely higher than conventionally.

### Example 7

### Effect of Rat Adipose Tissue-Derived Multipotent Stem Cells on Urine Incontinence

### 1. Experiment Method

To F344 female rat (body weight: about 150 g), F344 rat subcutaneous fat derived multipotent stem cells (3×10⁶) prepared by the method of Example 1 were expanded in a DMEM medium (Sigma) so that the total amount became 50 μl and injected in the bladder neck by using 30 Ginsulin injector (Mijector®). The thus treated rats were defined as a treatment group. On the other hand, 50 μl of DMEM instead of cell suspension was infused to rats in the control group. Two weeks after the infusion, the intravesical pressure was measured by the following method.

Firstly, rats in each group was anesthetized with urethane (0.8 g/kg, i.p.), and then, the spinal cord was cut at T8-9 level for the purpose of eliminating the urination reaction. After abdominal section, a catheter (PE-90) was retained in the bladder, the other end of the bladder catheter was connected to a reservoir of physiologic saline (60 ml syringe). The reservoir of physiologic saline was positioned at a certain height, so that the intravesical pressure was increased for 90 seconds. Thus, whether or not physiologic saline leaks out from the urethral meatus was observed. The intravesical pressure was increased each 2.5 cmH₂O. After 90-second observation time, the intravesical pressure was returned to 0 cmH₂O. Then, the following step was carried out. The intravesical pressure when the leakage of physiologic saline from the urethral meatus was observed was defined as a leak point pressure (LPP). LPP was measured three times repeatedly so as to calculate a mean value, which was made to be a representative value of each individual. LPP was measured before and after the excision of both sides of the pelvic nerve. The resultant values were compared between the treatment group (cell infusion group) and the control group (medium infusion group) by using Student's t-test.

On the other hand, after the LPP measurement, tissue specimen from the bladder neck was produced and subjected to HE staining and Masson trichrome staining.

### 2. Result

A significant difference (p<0.01) was observed between the treatment group and the control group before and after excision of the pelvic nerve (FIG. 26). That is to say, it is suggested that cell infusion increased the urethra internal pressure at least organically. This result suggests that the wall of the bladder neck is thickened in some way. Furthermore, it suggests the possibility of the increase of the pressure due to the thickening of wall, and the possibility of the differentiation into muscle and the increase of the muscular contraction power due to cytokines released from the cell.

On the other hand, as a result of HE staining (FIG. 27), in the treatment group (FIG. 27 left), at the position of 12:00 of the urethra, the formation of lump as agglomeration that is thought to be adipocyte was observed. As a result of Masson trichrome staining (FIG. 28), the most of the site where a lump is formed is composed of tissue that is thought to be collagen fibers composed of fibrous components (FIG. 28 left). As a result, it is suggested the possibility that adipose-derived multipotent stem cells produce collagen fibers.

### Example 8

### Effect of SVF Fraction on Renal Disorder 1

### 1. Experiment (Treatment) Protocol (FIG. 29)

(1) According to the method shown in Example 1, an SVF fraction was prepared from F344 rat subcutaneous fat.

(2) To an F344 rat (8-week age, male) whose one kidney had been extracted one week before, on day 0, cisplatin (7 mg/kg) was administered so as to form a cisplatin renal disorder rat (renal tubule necrosis model). On day 1, the SVF fraction (100 μl, 1×10⁶ cells) was subcapsularly infused (treatment group, 6 rats). To the control group (6 rats), the same amount of physiologic saline was administered under the same conditions.

(3) On days 0, 2, 4, 6 and 8 after the administration of cisplatin, the blood was collected and serum creatinine (Cr) value was measured.

(4) On day 4 after the administration of cisplatin, the renal blood flow in the capillary blood vessel around the renal tubule was measured by using a pencil type CCD camera.

### 2. Result

In the treatment group, on days 4 to 6 showing the peak of the cisplatin renal disorder, reduction of disorder was observed (FIG. 30. p<0.05 with respect to a control group). Thus, the therapeutic effect of the administration of an SVF fraction on renal disorder was observed.

On the other hand, the renal blood flow was significantly fast in the treatment group (p<0.01) (FIGS. 31-33).

### Example 9

### Effect of SVF Fraction on Renal Disorder 2

### 1. Experiment (Treatment) Protocol (FIG. 34)

(1) To the kidneys of an ischemia-reperfusion injury model produced by clamping both kidneys of a nude rat (8-week old, male) (IRI) for 30 minutes, SVF fraction (100 μl, 1×10⁶ cells) prepared from human adipose tissue by the method of Example 1 was directly infused (treatment group). To the control group, the same amount of physiologic saline was administered in the same conditions.

(2) On days 0, 1 and 2 after SVF infusion, the blood was collected and serum creatinine (Cr) value was measured.

### 2. Result

In the treatment group, on day 1 (p=0.053 with respect to control group) and on day 2 (p=0.075 with respect to control group), the serum creatinine value was reduced as compared with that of the control group. Thus, the reduction of the renal disorder was observed (FIG. 35).

### Example 10

### Effect of Mouse Adipose Tissue-Derived Multipotent Stem Cells on Osteoporosis

### 1. Experiment (Treatment) Protocol

(1) To an OCIF(OPG)KO mouse (9-week old, female), mouse adipose tissue-derived multipotent stem cells (100 μl, 1×10⁶ cells) prepared from a C57BL mouse (9-week old, female) according to the method shown in Example 1 was injected from caudal vein (OCIF treatment group). Furthermore, to OCIF(OPG)KO mouse, the same amount of phosphate buffer was administered in the same conditions (OCIF control group). Also to the C57BL mouse, the same amount of phosphate buffer was administered in the same conditions (C57BL control group).

(2) On days 0, 2, 4, 6, 8 and 10 after infusion of mouse adipose tissue-derived multipotent stem cells, the bone density of the thigh bone was measured.

### 2. Result

The OCIF treatment group shows the increase in the bone density from the early stage after cell administration and the increase over time in the bone density (FIG. 36). In the control group (OCIF control group and C57BL control group), the change in the bone density is not observed. This results show that the adipose tissue-derived multipotent stem cell is effective also for treatment of osteoporosis.

### Example 11

### Examination of Preparation Method of SVF Fraction

The sucked human subcutaneous fat (800 g) was divided into an equal amount (400 g each), and one of them was used in a preparation method in the below (1) and the other was used for the following method (2).

### (1) Conventional Method

Sucked fat (400 g) was treated with collagenase (37° C., 1 hour), followed by filtration using a filter having a hole diameter of 250-2000 μm. Subsequently, filtrate was centrifuged (1200 rpm, 5 minutes). The sediment was added to a medium to form an SVF fraction.

### (2) Improved Method

Sucked fat (400 g) was treated with collagenase (37° C., 1 hour) and then centrifuged (1200 rpm, 5 minutes). A medium was added to sediment so as to form a SVF fraction.

The SVF fraction obtained by the conventional method included 5.4×10⁷ cells. Meanwhile, the SVF fraction obtained by improved method included 1.12×10⁸ cells. Thus, as compared with the conventional method, the improved method was able to collect a larger number of cells. The improved method does not need filter process, thereby enabling SVF fraction to be obtained for a shorter time (about 1-2 hours, although depending upon the processing amount). In addition, a series of operations can be carried out in conditions near the closer system.

Next, in order to examine the therapeutic effect of the SVF fraction obtained by the improved method, a graft experiment using a cisplatin renal disorder rat was carried out. We employed the experimental protocol similar to that of Example 8 (effect of SVF fraction on renal disorder 1) (however, blood was collected on days 0, 2, 4 and 6) and compared the therapeutic effect of the SVF fraction obtained by the improved method with that of the SVF fraction obtained by the conventional method.

Experimental results (change of the serum creatinine value over time) are shown in FIG. 37. The SVF fraction obtained by the improved method exhibits the equal therapeutic effect to that of the SVF fraction obtained by the conventional method.

### Example 12

### Examination of Resistance of SVF Fraction to Freezing/Thawing

We examined whether or not the cell proliferation potency, cytokine secretion capacity and surface antigen of SVF fraction are changed by freezing/thawing process.

### 1. Experiment Method

A SVF fraction prepared by the method in Example 10(1) was transferred to −80° C. deep freezer and frozen. On day 30, it was transferred to 37° C. incubator and thawed. The cell proliferation potency and the cytokine secretion capacity of the SVF fraction that had undergone the freezing/thawing process (hereinafter, “freezing-processed SVF fraction”) were compared with those of a control SVF fraction (SVF fraction that had not undergo a freezing/thawing process). Furthermore, the cell surface antigen of the freezing-processed SVF fraction was analyzed by FACS.

### 2. Result

No difference in the cell proliferation potency was observed between the freezing-processed SVF fraction and the control SVF fraction (FIG. 38). Also, no difference in cytokines (VEGF-A and VEGF-C) secretion capacity was observed between the freezing-processed SVF fraction and the control SVF fraction (FIGS. 39 and 40). Cell surface antigens (CD34 and CD13) of the freezing-processed SVF fraction were similar to those of the SVF fraction that had been reported to date (FIG. 41).

From the above-mentioned results, it was clarified that the SVF fraction had high resistance to the freezing/thawing process.

### INDUSTRIAL APPLICABILITY

A cell preparation of the present invention is used for treating ischemia disease, renal dysfunction or wound. According to the cell preparation of the present invention, an excellent effect of reconstructing tissue by multipotent cells derived from adipose tissue as an effective component is obtained. By using adipose tissue as a cell source, it is possible to obtain a necessary amount of cells without giving an excessive burden to a patient. Therefore, the present invention provides a cell preparation that gives few burdens to a patient.

In one embodiment of the cell preparation of the present invention, cells proliferated by a low-serum culture are used. Since the low-serum culture uses a small amount of serum, without using the serum from a heterogeneous animal, a necessary amount of the serum can be secured. That is to say, cells of the present invention can be obtained by using only serum of patients themselves (or heterogeneous serum as needed). Therefore, in this embodiment, it is possible to provide a cell preparation having high safety, which has been obtained by a manufacturing process excluding heterogeneous animal materials.

The present invention is not limited to the descriptions of Embodiments and Examples of the above-described invention at all. The present invention also includes a variety of modified aspects in the scope where those skilled in the art can easily conceive without departing the scope of the claims.

Each of the theses, Publication of Patent Applications, Patent Publications, and other published documents mentioned or referred to in this specification is herein incorporated by reference in its entity.

### BRIEF DESCRIPTION OF THE DRAWINGS

FIG. 1 is a graph showing a comparison of the change over time of the lower limb cumulative survival rate (by Kaplan-Meier method) between a group of mouse lower limb ischemia model to which human adipose tissue-derived multipotent stem cells were infused (treatment group) and a control group.

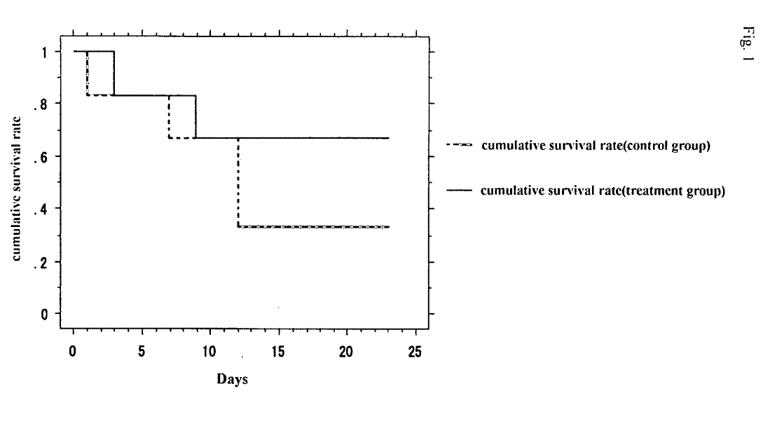

FIG. 2 shows states of both models (representative examples) on day 7 after treatment. In the control group on the left, black necrosis in the left lower limb is observed, while the treatment group on the right shows ruddy complexion.

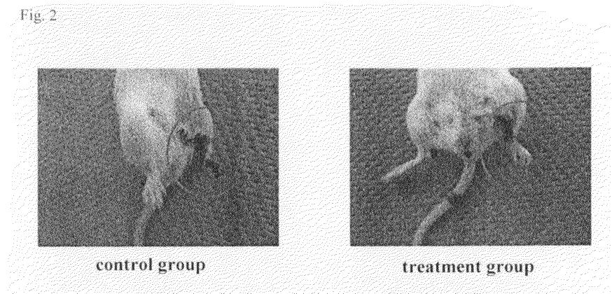

FIG. 3 shows properties of a rat renal failure model (folic acid renal failure model) used in Example. A left view is a graph showing the change over time of the blood urea nitrogen amount in the model. A right view shows a PAS-stained image of renal tissue collected on day 1 after folic acid was administered.

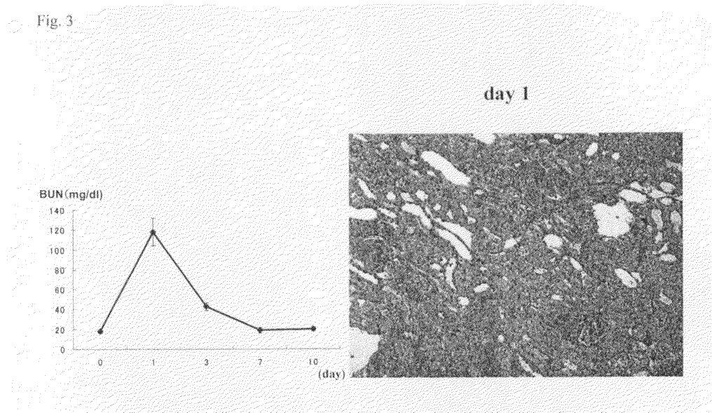

FIG. 4 is a graph showing a comparison of the change over time of the blood urea nitrogen amount between a group of rat renal failure model to which adipose tissue-derived multipotent stem cells were infused (treatment group) and a control group.

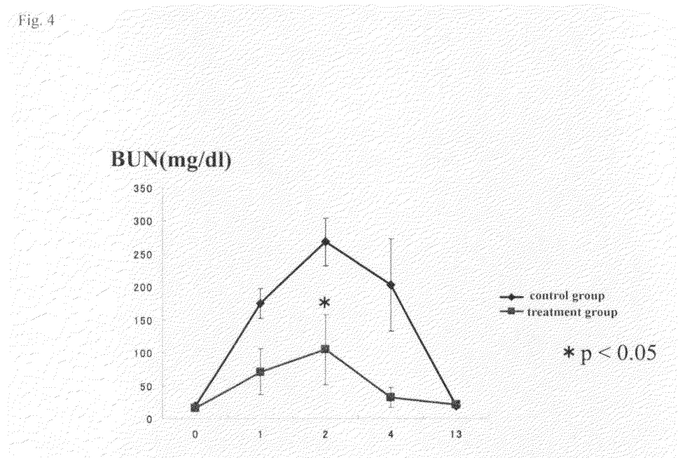

FIG. 5 shows states of the renal tissue of a rat renal failure model on day 13 after treatment (PAS-stained image). In the control group on the left, expansion of the renal tubule and deciduation of the renal tubule epithelium cells are observed. In the treatment group on the right, such images are hardly observed, which resembles the normal tissue.

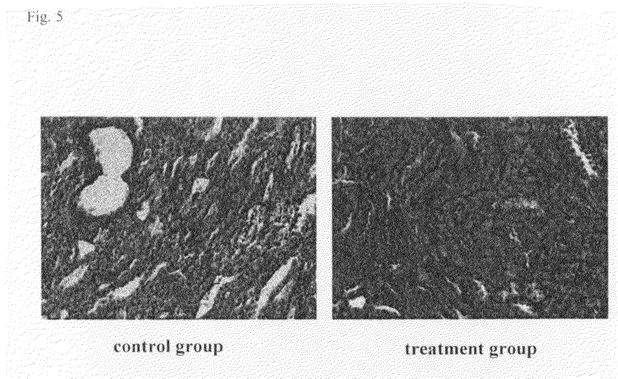

FIG. 6 shows states of the renal tissue of a rat renal failure model on day 13 after treatment (Masson trichrome-stained image). In the control group on the left, the atrophy of the renal tubule and fibrosis of the interstitial tissue are observed. In the treatment group on the right, such images are hardly observed, which resembles the normal tissue.

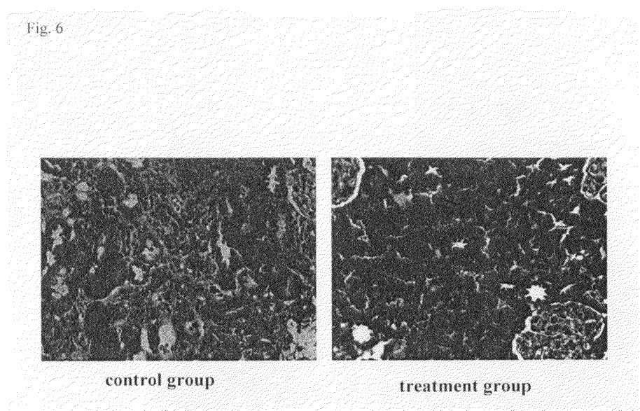

FIG. 7 schematically shows a method of measuring a blood flow of the capillary blood vessel around the renal tubule.

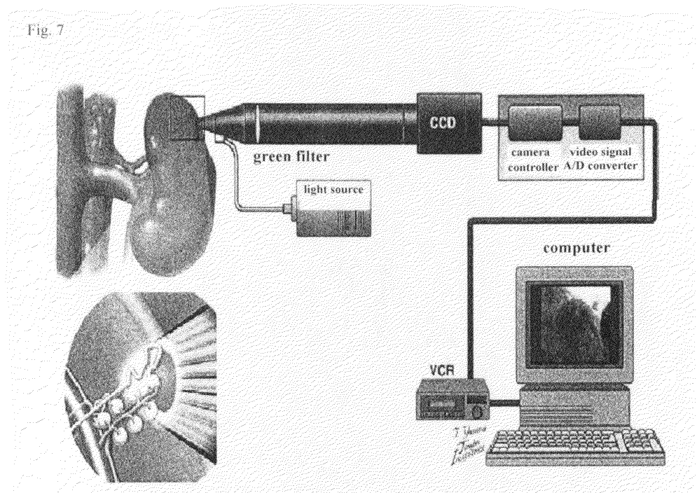

FIG. 8 is a view showing a blood flow of the capillary blood vessel around the renal tubule (control group).

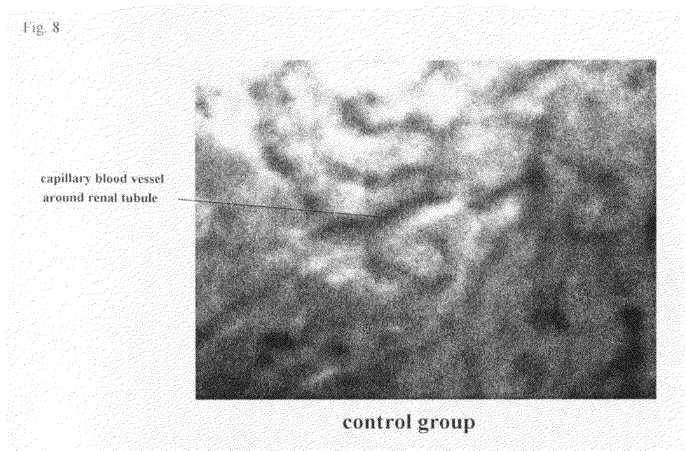

FIG. 9 is a view showing a blood flow of the capillary blood vessel around the renal tubule (treatment group).

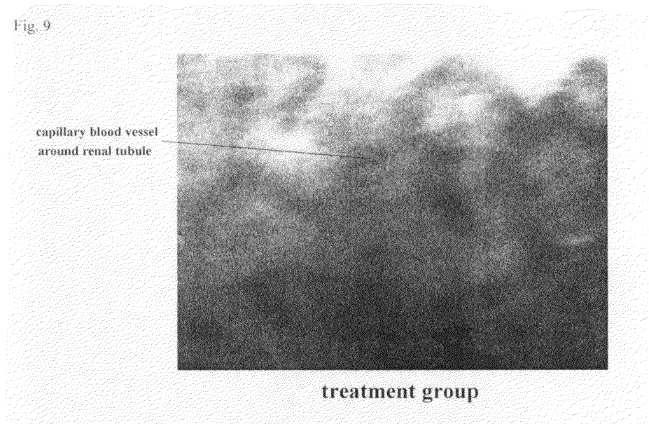

FIG. 10 is a graph showing a comparison of the change over time of the blood urea nitrogen amount between a group of rat renal failure model to which human adipose tissue-derived multipotent stem cells were infused (treatment group) and a control group.

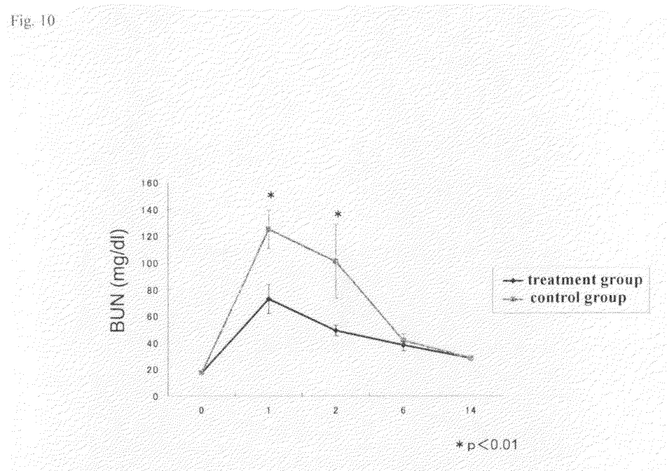

FIG. 11 is a view (immunostaining image) showing a state of the renal tissue of the rat renal failure model on day 14 after treatment. The movement of the administered cells into the parenchyma of kidney is not observed and the cells are survived under the renicapsule.

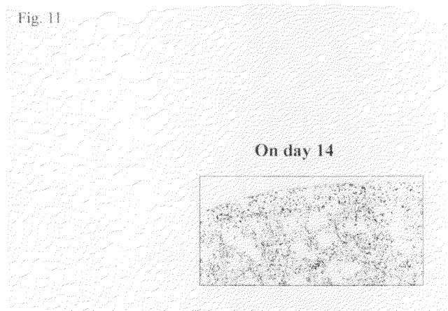

FIG. 12 is a view (immunostaining image) showing a state of the renal tissue of the rat renal failure model one month after treatment. The administered cells remain under the renicapsule.

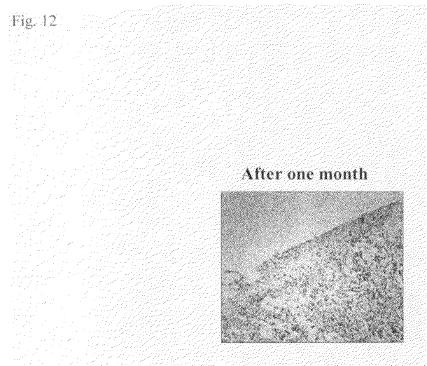

FIG. 13 is a view (immunostaining image) showing a state of the renal tissue of the rat renal failure model three months after treatment. The administered cells remain under the renicapsule.

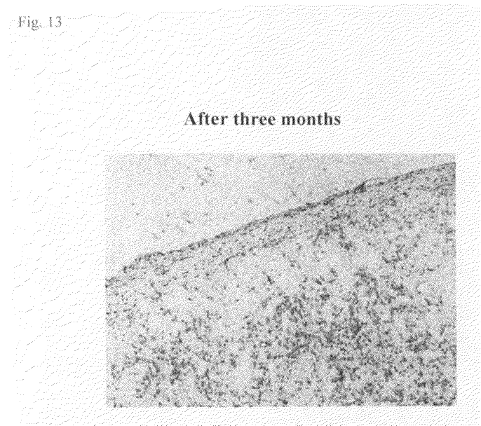

FIG. 14 is a graph showing a comparison of a blood flow of the capillary blood vessel around renal tubule between a group of rat renal failure model to which human adipose tissue-derived multipotent stem cells was infused and the control group.

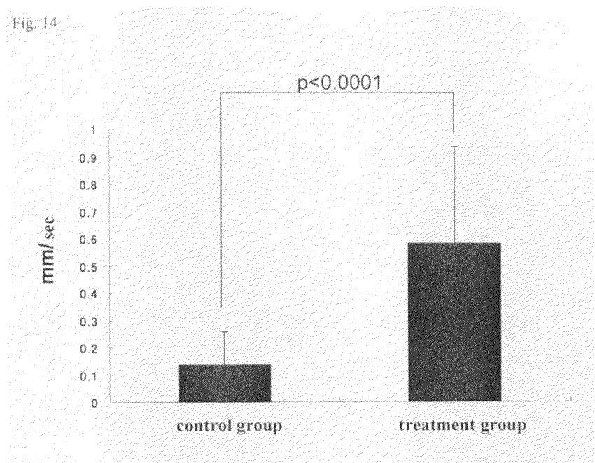

FIG. 15 shows a production protocol of a rat skin defect model.

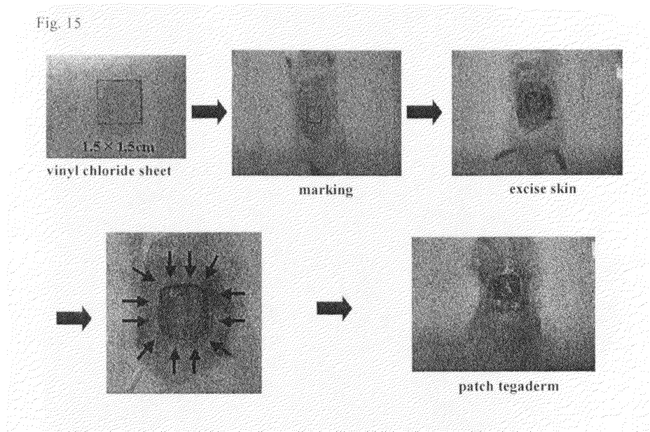

FIG. 16 is a graph showing a comparison of the change over time of the skin defect area among a group of rat skin defect model to which the rat adipose tissue-derived multipotent stem cells were infused (low-serum treatment group), a group to which cells cultured in the high-serum conditions were infused (high-serum treatment group) and a control group.

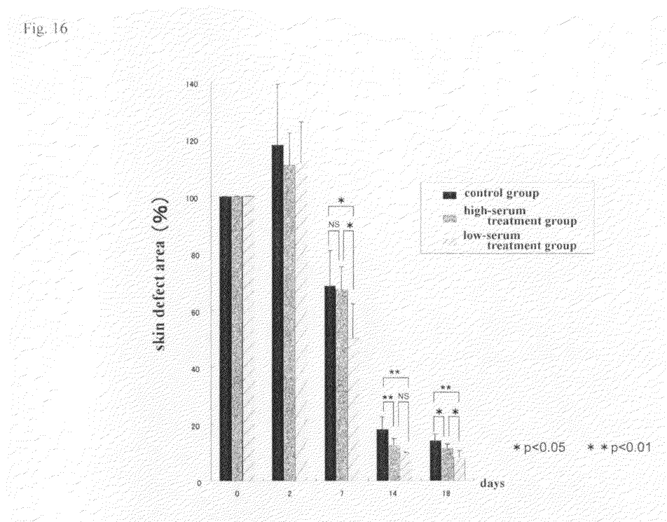

FIG. 17 shows states of a wounded portion of a rat skin defect model on day 14 after treatment. In the low-serum treatment group (right upper picture), the rapid hearing of the skin defect area was observed as compared with the control group (left upper picture). Furthermore, in the low-serum treatment group, the state of the scar tissue is excellent. The effect of promoting healing wound in the low-serum treatment group is higher as compared with that of the high-serum treatment group (left lower picture).

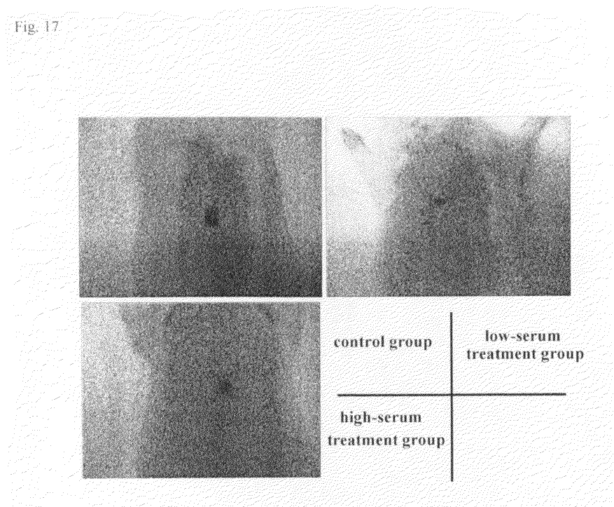

FIG. 18 shows the cytokine concentration in skin tissue three days after the treatment. As shown in the upper part, the cytokine concentration is carried out among the brood bud, inside of marginal region and outside of marginal region. Lower left graph shows a comparison of VEGF concentration; lower right graph shows a comparison of HGF concentration.

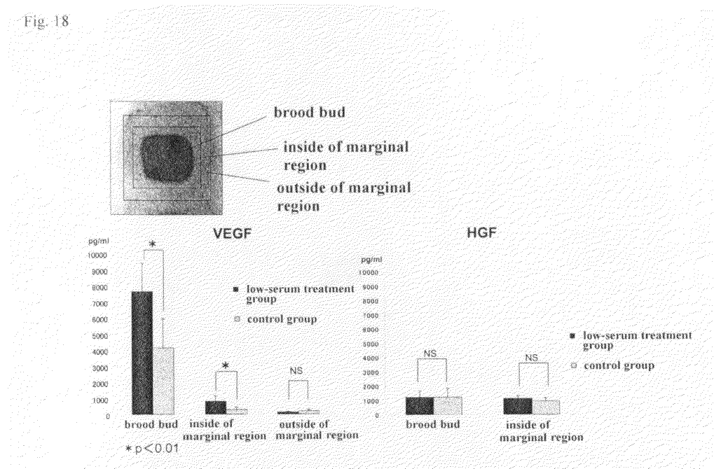

FIG. 19 shows a comparison of secretion amounts of various cytokines. The cells obtained by culturing SVF fraction derived from human adipose tissue under low-serum conditions (low-serum culture group) show larger secretion amounts of VEGF-A, HGF, VEGF-C and FGF-7(KGF) as compared with the control group (HEK293).

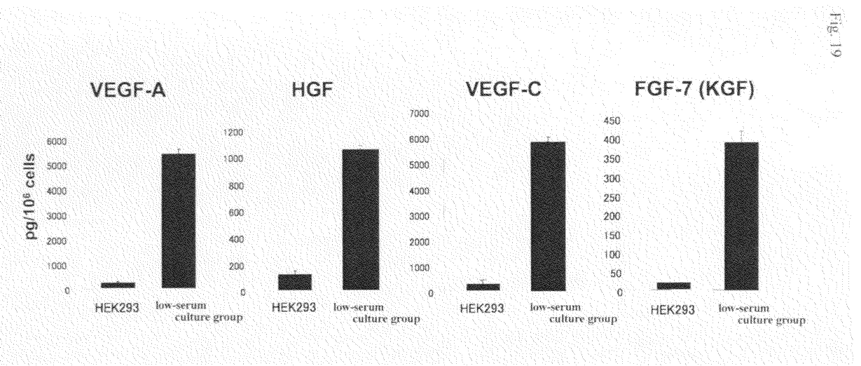

FIG. 20 shows a comparison of FGF-2 secretion amount. The low-serum culture group secretes a larger amount of FGF-2 than the control group (HEK293).

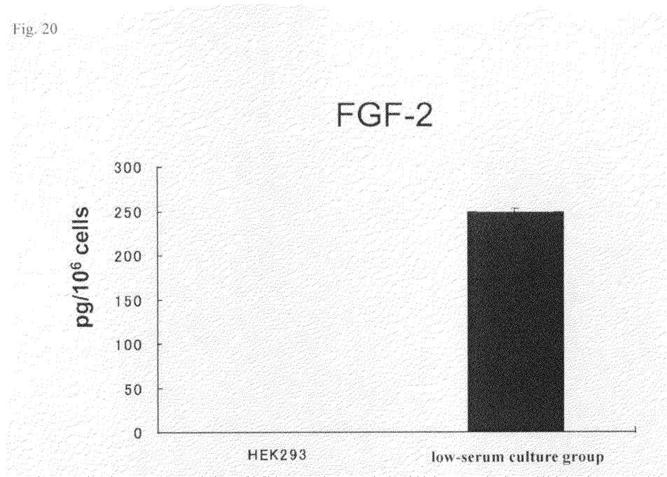

FIG. 21 shows a comparison of VEGF-A secretion amount. The low-serum culture group exhibits a larger VEGF-A secretion amount as compared with the high-serum culture group and bFGF-added high-serum cultured group.

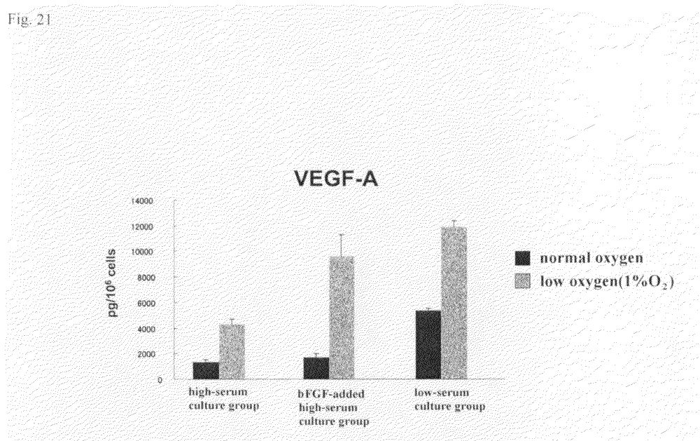

FIG. 22 shows a comparison of FGF-7(KGF) secretion amount. The low-serum culture group exhibits a larger FGF-7(KGF) secretion amount as compared with the high-serum culture group and bFGF-added high-serum cultured group.

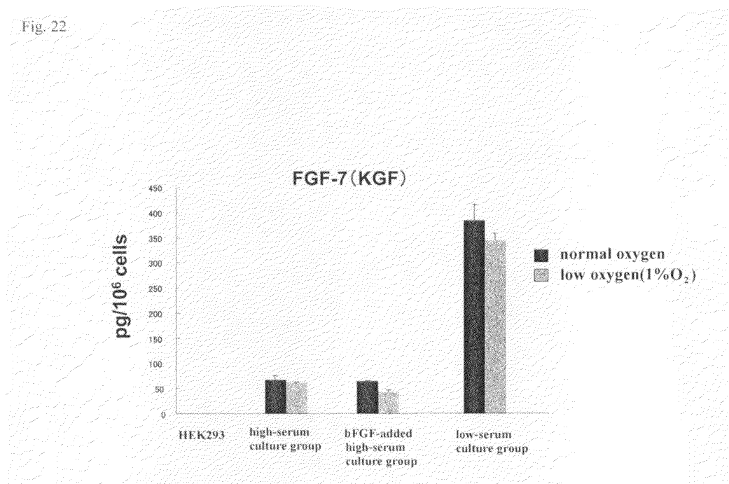

FIG. 23 shows a comparison of FGF-2 secretion amount. The low-serum culture group exhibits a larger FGF-2 secretion amount as compared with the high-serum culture group and bFGF-added high-serum cultured group.

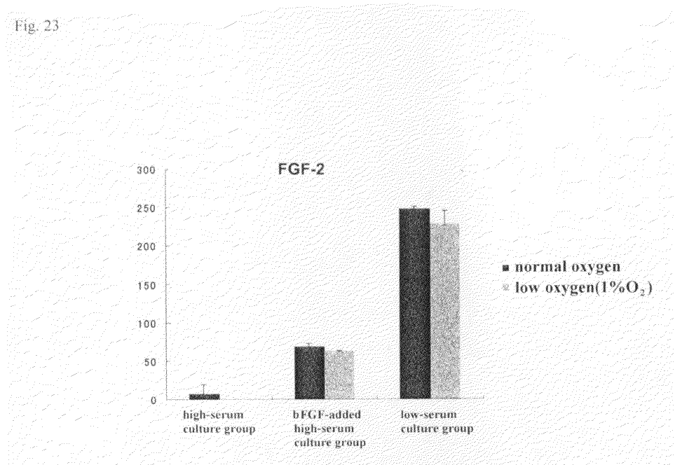

FIG. 24 shows a comparison of secretion amounts of VEGF-C and HGF. A remarkable difference in the VEGF-C secretion amount and the HGF secretion amount between the groups is not observed.

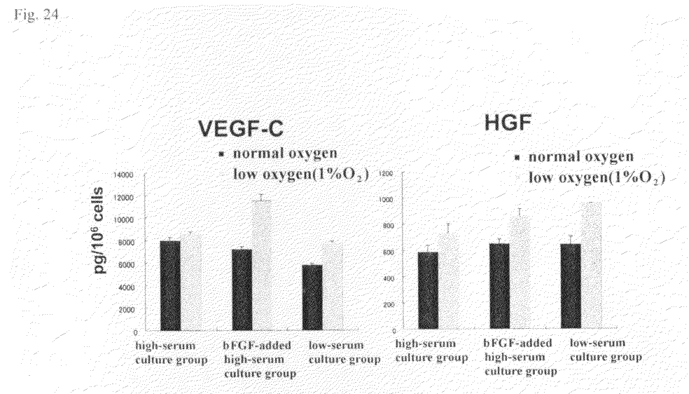

FIG. 25 shows a comparison of the secretion amounts of TGF-β, IL-6, IL-10 and IL-8. The low-serum culture group shows a larger secretion amounts of TGF-β, IL-6, IL-10 and IL-8 as compared with the high-serum group and the bFGF-added high-serum cultured group.

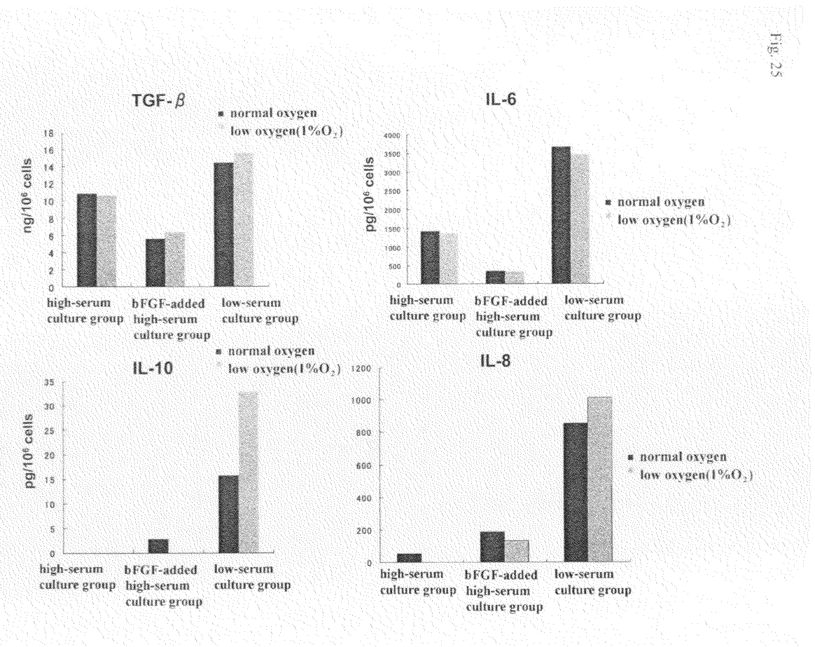

FIG. 26 shows an effect of rat adipose tissue-derived multipotent stem cells on urine incontinence. A pressure at the leakage time is compared before and after the excision of the pelvic nerve between the treatment group (cell administered group) and the control group. Mean±standard deviation. N=7, **p<0.01 (by Student's t-test).

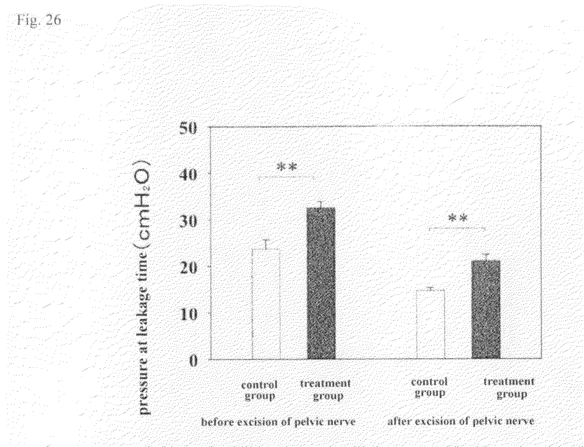

FIG. 27 shows an effect of rat adipose tissue-derived multipotent stem cells on the urine incontinence. HE stained images of the bladder neck are shown. A left picture shows a treatment group (magnification of upper part: ×400 times, magnification of lower part: ×50) and right picture shows the control group (magnification: ×50).

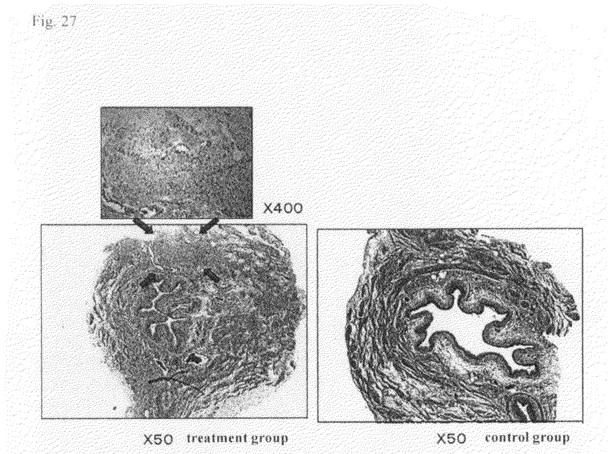

FIG. 28 shows an effect of rat adipose tissue-derived multipotent stem cells on the urine incontinence. Masson trichrome-stained images of the bladder neck are shown. A left picture shows a treatment group (magnification of upper picture: ×400, magnification of lower picture: ×50) and right picture shows the control group (magnification: ×50).

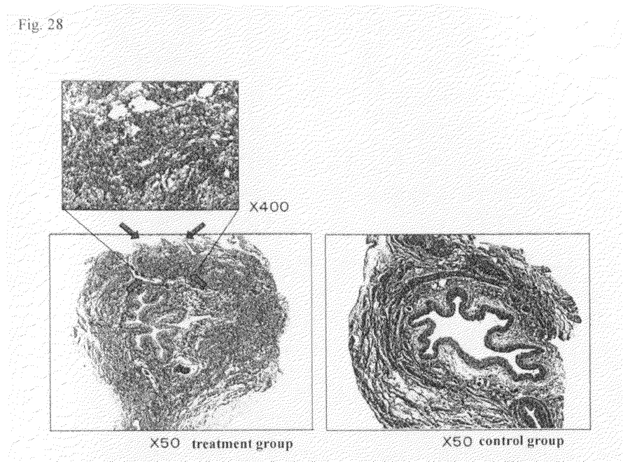

FIG. 29 shows a protocol of an experiment using a cisplatin renal disorder model.

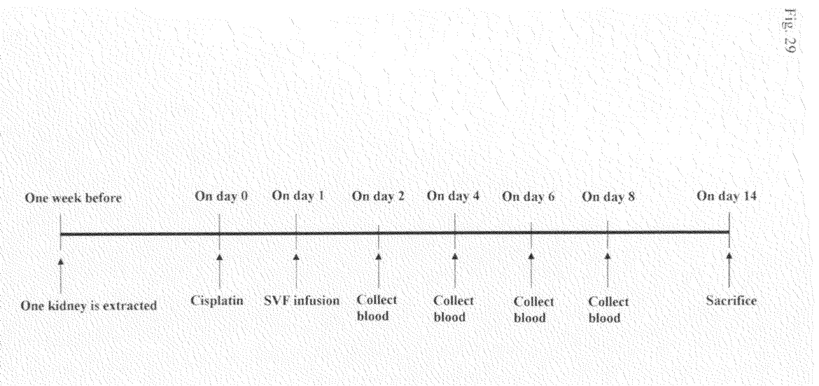

FIG. 30 is a graph showing a comparison of the serum creatinine value between a treatment group (SVF fraction is administered to cisplatin renal disorder model) and a control group.

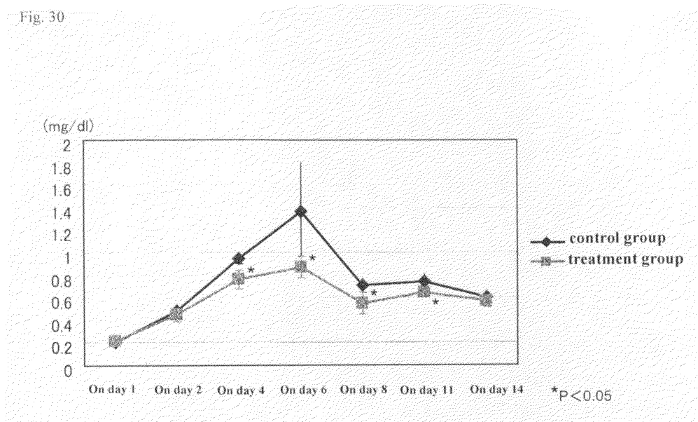

FIG. 31 shows a renal blood flow (control group).

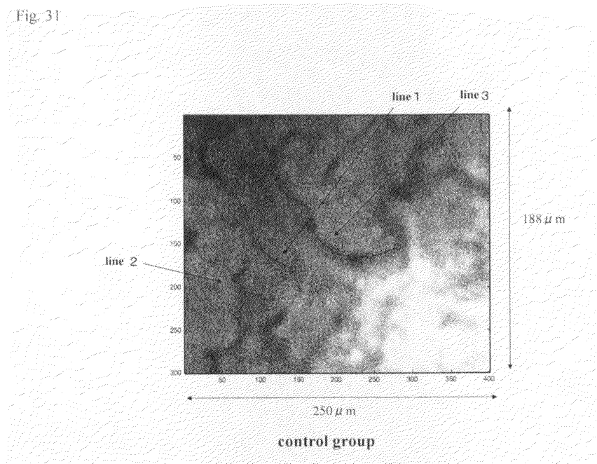

FIG. 32 shows a renal blood flow (treatment group).

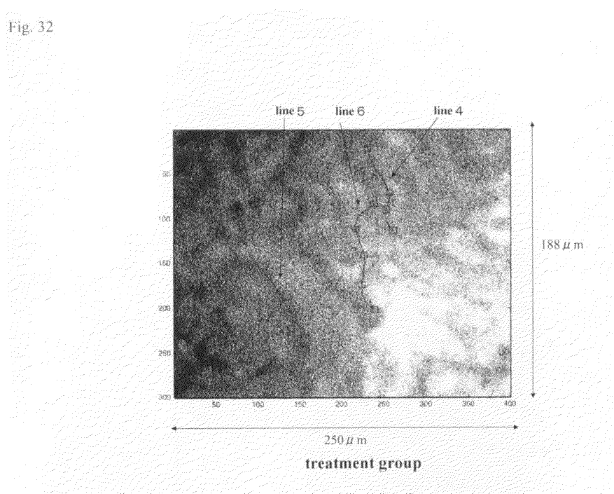

FIG. 33 shows a comparison of a renal blood flow between the control group and the treatment group.

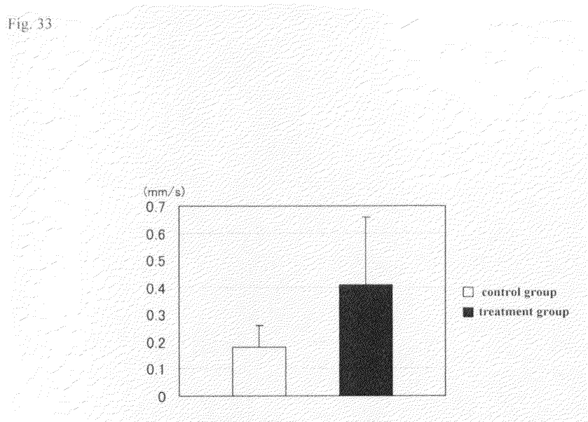

FIG. 34 shows a protocol of an experiment using an ischemia-reperfusion injury model.

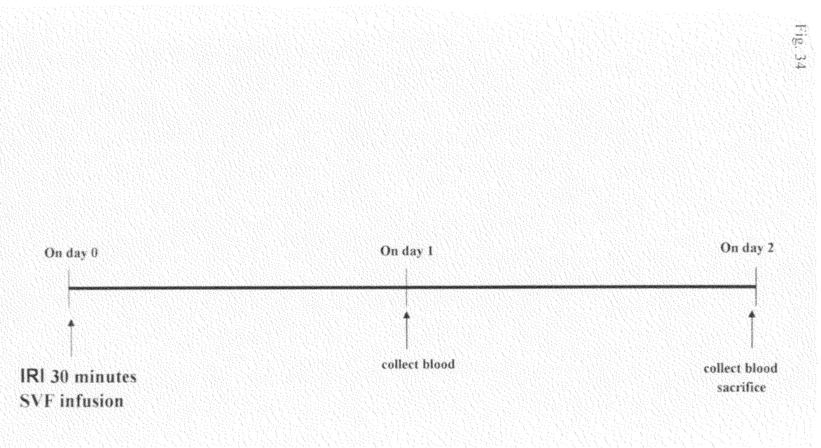

FIG. 35 is a graph showing a comparison of the serum creatinine value between the treatment group (SVF fraction is administered to an ischemia-reperfusion injury model) and the control group.

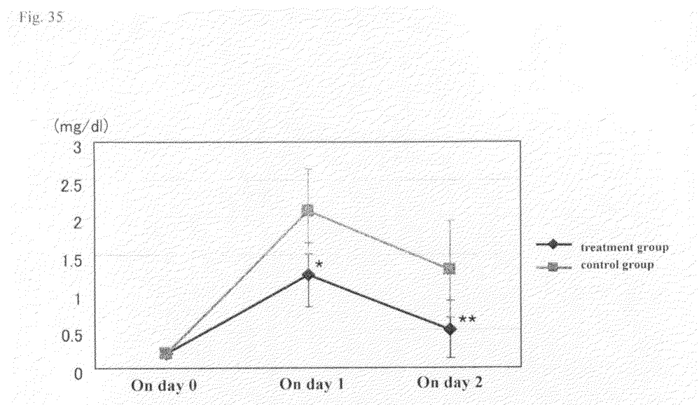

FIG. 36 shows an effect of adipose tissue-derived multipotent stem cells on osteoporosis. To an OCIF(OPG)KO mouse as an osteoporosis model, mouse adipose tissue-derived multipotent stem cells were injected from caudal vein (OCIF treatment group), and then, the change over time of the bone density of the thigh bone was examined. To an OCIF control group, the same amount of phosphate buffer was injected from the caudal vein. Furthermore, also to the C57BL mouse, the same amount of phosphate buffer was injected from caudal vein (C57BL control group).

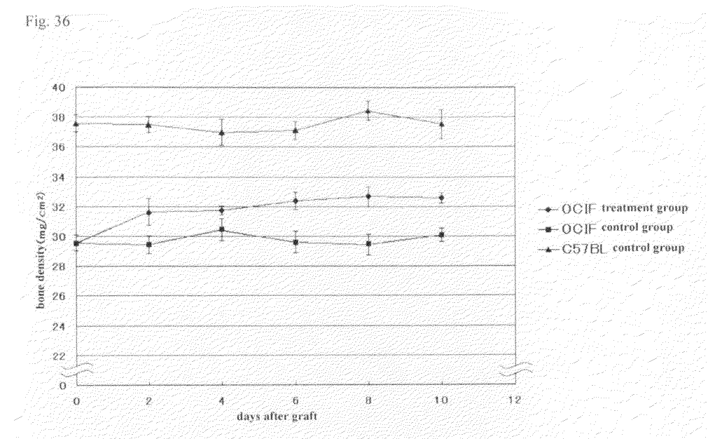

FIG. 37 shows an effect of the SVF fraction obtained by an improved method on the renal disorder. The SVF fraction obtained by an improved method is administered to a cisplatin renal disorder rat (rSVF improved method), and the change over time of the serum creatinine value is compared with the case of the SVF fraction obtained by a conventional method is administered (rSVF conventional method). To a control group, instead of the cells, the same amount of physiologic saline was administered.

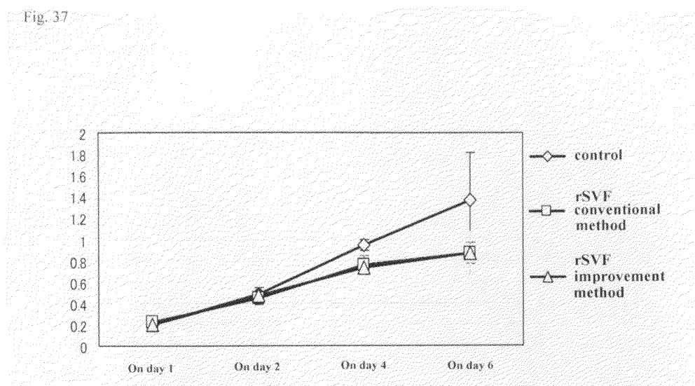

FIG. 38 is a graph showing a comparison in cell proliferation potency between the SVF fraction undergoing the freezing/thawing process (freezing-processed SVF fraction) and the control SVF fraction.

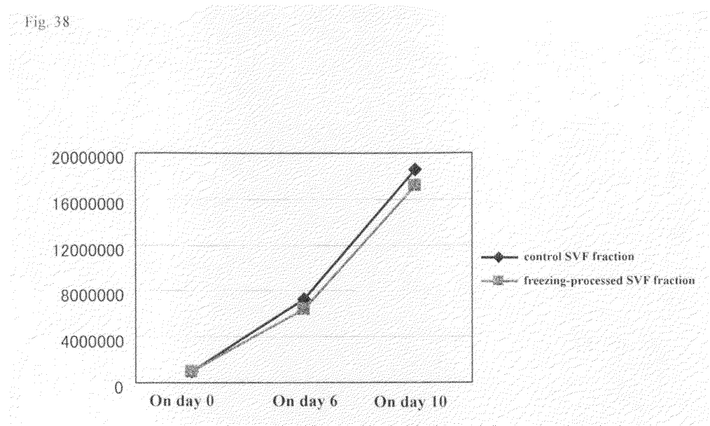

FIG. 39 is a graph showing a comparison in secretion capacity of cytokine (VEGF-A) between the SVF fraction undergoing the freezing/thawing process (freezing-processed SVF fraction) and the control SVF fraction. The freezing-processed SVF fraction has the equal level of VEGF-A secretion capacity to that of the control SVF fraction.

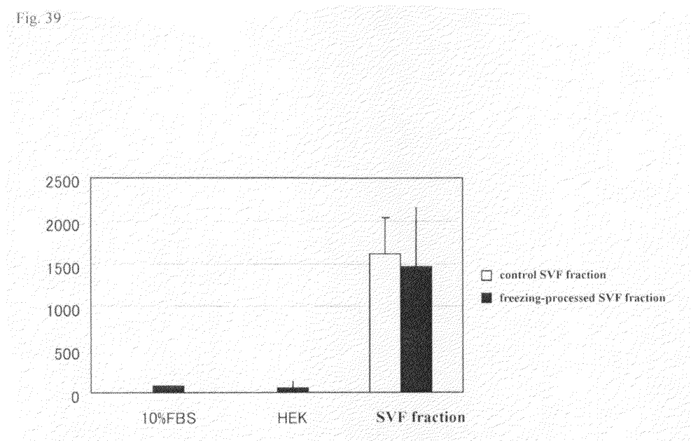

FIG. 40 is a graph showing a comparison in secretion capacity of cytokine (VEGF-C) between the SVF fraction undergoing the freezing/thawing process (freezing-processed SVF fraction) and the control SVF fraction. The freezing-processed SVF fraction has an equal level of VEGF-C secretion capacity to that of the control SVF fraction. The freezing-processed SVF fraction has the same VEGF-C secretion capacity as that of the control SVF fraction. In any of the freezing-processed SVF fraction and the control SVF fraction, the reduction of VEGF-C secretion capacity due to the low-oxygen culture is observed.

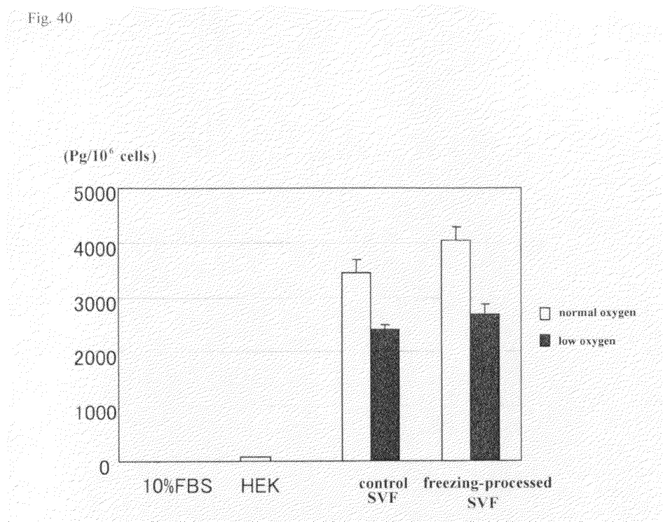

FIG. 41 is a graph showing the FACS analysis results of the cell surface antigen of the SVF fraction that was undergone the freezing/thawing process, showing the same CD34 positive rate (left) and CD13 positive rate (right) as those in the past report.

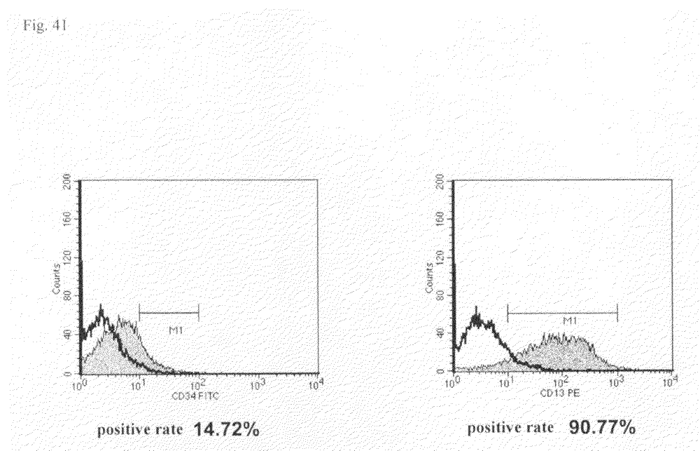

1. A cell preparation which contains CD34-negative, CD90-positive and CD117-negative adipose tissue-derived multipotent stem cells that are proliferated when a cell population separated from adipose tissue is cultured in low-serum conditions, and which is usable for ischemia disease, renal dysfunction, wound, urine incontinence or osteoporosis.

2. The cell preparation of claim 1, wherein the adipose tissue-derived multipotent stem cells are cells proliferated when a sedimented cell population, which is sedimented when a cell population separated from adipose tissue is centrifuged at 800-1500 rpm for 1-10 minutes, is cultured under low-serum conditions.

3. The cell preparation of claim 1, wherein the low-serum conditions are conditions in which a serum concentration in the culture solution is 5% (V/V) or less.

4. The cell preparation of claim 1, wherein the sedimented cell population is a sedimented cell population (a) or (b):
(a) a sedimented cell population collected as sediments by treating adipose tissue with protease, then subjecting the cell population to filtration, and then centrifuging the filtrate;
(b) a sedimented cell population collected as sediments by treating adipose tissue with protease, and then centrifuging adipose tissue without filtration.

5. The cell preparation of claim 4, wherein the protease is collagenase.

6. The cell preparation of claim 4, wherein the centrifugation is carried out under conditions at 800-1500 rpm for 1-10 minutes.

7. The cell preparation of claim 1, wherein the adipose tissue is human adipose tissue.

8. The cell preparation of claim 1, which is in a frozen state.

9. A use of CD34-negative, CD90-positive and CD117-negative adipose tissue-derived multipotent stem cells for producing a cell preparation for ischemia disease, renal dysfunction, wound, urine incontinence or osteoporosis.

10. A treatment method comprising: administering CD34-negative, CD90-positive and CD117-negative adipose tissue-derived multipotent stem cells to a patient with ischemia disease, renal dysfunction, wound, urine incontinence or osteoporosis.

11-14. (canceled)
